SEQUENCE LISTING (15 sequences)

SEQ ID NO: 1 - DNA, 867 bp, Homo sapiens
atgcagatgtgtggagccggcttgcccgttgctggatctgggaccaacggaggtagtcttgtcttgggtggaattgaaccaagtttgtataaaggagacatctggtatacccctattaaggaagagtggtactaccagatagaaattctgaaattggaaattggaggccaaagccttaatctggactgcagagagtataacgcagacaaggccatcgtggacagtggcaccacgctgctgcgcctgccccagaaggtgtttgatgcggtggtggaagctgtggcccgcgcatctctgattccagaattctctgatggtttctggactgggtcccagctggcgtgctggacgaattcggaaacaccttggtcttacttccctaaaatctccatctacctgagagacgagaactccagcaggtcattccgtatcacaatcctgcctcagctttacattcagcccatgatgggggccggcctgaattatgaatgttaccgattcggcatttccccatccacaaatgcgctggtgatcggtgccacggtgatggagggcttctacgtcatcttcgacagagcccagaagagggtgggcttcgcagcgagcccctgtgcagaaattgcaggtgctgcagtgtctgaaatttccgggcctttctcaacagaggatgtagccagcaactgtgtccccgctcagtctttgagcgagcccattttgtggattgtgtcctatgcgctcatgagcgtctgtggagccatcctccttgtcttaatcgtcctgctgctgctgccgttccggtgtcagcgtcgcccccgtgaccctgaggtcgtcaatgatgagtcctctctggtcagacatcgctggaaa

SEQ ID NO: 2 - protein, 289 aa, Homo sapiens
MQMCGAGLPVAGSGTNGGSLVLGGIEPSLYKGDIWYTPIKEEWYYQIEILKLEIGGQSLNLDCREYNADKAIVDSGTTLLRLPQKVFDAVVEAVARASLIPEFSDGFWTGSQLACWTNSETPWSYFPKISIYLRDENSSRSFRITILPQLYIQPMMGAGLNYECYRFGISPSTNALVIGATVMEGFYVIFDRAQKRVGFAASPCAEIAGAAVSEISGPFSTEDVASNCVPAQSLSEPILWIVSYALMSVCGAILLVLIVLLLLPFRCQRRPRDPEVVNDESSLVRHRWK

SEQ ID NO: 3 - DNA, 1038 bp, Homo sapiens
atgggcgcactggcccgggcgctgctgctgcctctgctggcccagtggctcctgcgcgccgccccggagctggcccccgcgcccttcacgctgcccctccgggtggccgcggccacgaaccgcgtagttgcgcccaccccgggacccgggacccctgccgagcgccacgccgacggcttggcgctcgccctggagcctgccctggcgtcccccgcgggcgccgccaacttcttggccatggtagacaacctgcagggggactctggccgcggctactacctggagatgctgatcgggacccccccgcagaagctacagattctcgttgacactggaagcagtaactttgccgtggcaggaaccccgcactcctacatagacacgtactttgacacagagaggtctagcacataccgctccaagggctttgacgtcacagtgaagtacacacaaggaagctggacgggcttcgttggggaagacctcgtcaccatccccaaaggcttcaatacttcttttcttgtcaacattgccactatttttgaatcagagaatttctttttgcctgggattaaatggaatggaatacttggcctagcttatgccacacttgccaagccatcaagttctctggagaccttcttcgactccctggtgacacaagcaaacatccccaacgttttctccatgcagatgtgtggagccggcttgcccgttgctggatctgggaccaacggaggtagtcttgtcttgggtggaattgaaccaagtttgtataaaggagacatctggtatacccctattaaggaagagtggtactaccagatagaaattctgaaattggaaattggaggccaaagccttaatctggactgcagagagtataacgcagacaaggccatcgtggacagtggcaccacgctgctgcgcctgccccagaaggtgtttgatgcggtggtggaagctgtggcccgcgcatctctgaaattgcaggtgctgcagtgtctgaaatttccgggcctttctcaacagaggatg

SEQ ID NO: 4 - protein, 346 aa, Homo sapiens
MGALARALLLPLLAQWLLRAAPELAPAPFTLPLRVAAATNRVVAPTPGPGTPAERHADGLALALEPALASPAGAANFLAMVDNLQGDSGRGYYLEMLIGTPPQKLQILVDTGSSNFAVAGTPHSYIDTYFDTERSSTYRSKGFDVTVKYTQGSWTGFVGEDLVTIPKGFNTSFLVNIATIFESENFFLPGIKWNGILGLAYATLAKPSSSLETFFDSLVTQANIPNVFSMQMCGAGLPVAGSGTNGGSLVLGGIEPSLYKGDIWYTPIKEEWYYQIEILKLEIGGQSLNLDCREYNADKAIVDSGTTLLRLPQKVFDAVVEAVARASLKLQVLQCLKFPGLSQQRM

SEQ ID NO: 5 - DNA, 20 bp, Homo sapiens
cgcatctctgaaattgcagg

SEQ ID NO: 6 - protein, 10 aa, Homo sapiens
MQMCGAGLPV

SEQ ID NO: 7 - protein, 10 aa, Homo sapiens
ARASLKLQVL

SEQ ID NO: 8 - DNA, 25 bp, Homo sapiens
gtctaccctggtaccgcctttcttt

SEQ ID NO: 9 - DNA, 25 bp, Homo sapiens
ctccacacatctgcatggagaaaac

SEQ ID NO: 10 - DNA, 26 bp, Homo sapiens
tataacgcagacaaggccatcgtgga

SEQ ID NO: 11 - DNA, 28 bp, Homo sapiens
gcagcaggacgattaagacaaggaggat

SEQ ID NO: 12 - DNA, 27 bp, Homo sapiens
atgcagatgtgtggagccggcttgccc

SEQ ID NO: 13 - DNA, 27 bp, Homo sapiens
tcatttccagcgatgtctgaccagaga

SEQ ID NO: 14 - DNA, 27 bp, Homo sapiens
atgggcgcactggcccgggcgctgctg

SEQ ID NO: 15 - DNA, 27 bp, Homo sapiens
ctacatcctctgttgagaaaggcccgg

(19) United States
(12) Patent Application Publication
(10) Pub. No.: US 2004/0203042 A1
(43) Pub. Date: Oct. 14, 2004

(54) Alternatively spliced isoforms of aspartyl protease 1 (BACE2)

(76) Inventors: Jason M. Johnson, Seattle, WA (US); Philip W. Garrett-Engele, Seattle, WA (US)

Correspondence Address: R. Douglas Bradley, Merck & Co., Inc., Patent Department RY60-30, P.O. Box 2000, Rahway, NJ, 07065-0907, US

(21) Appl. No.: 10/791,488

(22) Filed: Mar. 2, 2004

Related U.S. Application Data
(60) Provisional application No. 60/452,292, filed on Mar. 4, 2003.

(51) Int. Cl. C12Q 1/68; C07H 21/04; C12N 9/64

(52) U.S. Cl. 435/6; 435/69.1; 435/226; 435/320.1; 435/325; 536/23.2

(57) ABSTRACT

The present invention features nucleic acids and polypeptides encoding two novel splice variant isoforms of aspartyl protease 1 (BACE2). The polynucleotide sequences of BACEsv1 and BACE2sv2 are provided by SEQ ID NO 1 and SEQ ID NO 3, respectively. The amino acid sequences for BAC2sv1 and BACE2sv2 are provided by SEQ ID NO 2 and SEQ ID NO 4, respectively. The present invention also provides methods for using BACE2sv1 and BACE2sv2 polynucleotides and proteins to screen for compounds that bind to BACE2sv1 and BACE2sv2, respectively.

This application claims priority to U.S. Provisional Patent Application Serial No. 06/452,292 filed on March 04, 2003, which is incorporated by reference herein in its entirety.

### BACKGROUND OF THE INVENTION

The references cited herein are not admitted to be prior art to the claimed invention.

One of the major characteristics of Alzheimer's disease (AD) is the accumulation in the brain of sticky plaques and vascular deposits consisting of the insoluble 4-kD amyloid β peptide (Aβ) (Vassar, et. al., 1999, Science 286, 735-741). Amyloid β is formed when the transmembrane β-amyloid precursor protein (APP) is cleaved by proteases. Three distinct enzymes are involved in the processing of APP: α-, β-, and γ- secretase. Amyloid β formation is initiated when β-secretase cleaves APP at the amino terminus, forming a soluble amino-terminal fragment and a membrane bound carboxy-terminal fragment. The carboxy terminal fragment is cleaved from the membrane by γ-secretase, resulting in the release of insoluble amyloid β (Vassar, et. al., 1999, Science 735-741). In an alternative pathway, α-secretase cleaves APP within the amyloid β domain, forming a soluble protein as well as a membrane-bound peptide. When the membrane-bound peptide is cleaved by y-secretase, the result is a nontoxic protein (Bennett, et. al., 2000, J of Biol. Chem. 275, 20647-20651).

The β-secretase enzyme has been identified as BACE (β-site APP cleaving enzyme) (Vassar, et. al., 1999, Science 286, 735-741; Sinha, et. al., 1999, Nature 402, 537-540). A BACE homolog, BACE2, has also been identified (Saunders, et. al., 1999, Science 286, 1255a; Acquati, et. al., 2000, FEBS 468 59-64). BACE2 has 52% amino acid homology and 68% similarity with BACE. The two protease active sites are conserved between the two genes and both BACE and BACE2 are transmembrane aspartyl proteases, unusual in that other known aspartyl proteases are soluble molecules. While BACE maps to chromosome 11, BACE2 maps to the Down syndrome region of chromosome 21. This could be significant because middle-aged Down syndrome (DS) patients also exhibit accumulation of amyloid β deposits in the brain. Since DS patients have three copies of chromosome 21, they would be expected to express larger amounts of APP as well as BACE2 enzyme, as compared to the general population with two copies of chromosome 21 (Saunders, et. al., 1999, Science 286, 1255a; Acquati, et. al., 2000, FEBS 468 59-64).

Whereas messenger RNA of BACE is expressed at high levels in the brain, mRNA levels of BACE2 are very low or undetectable in brain (Bennett, et. al., 2000, J. of Biol. Chem. 275, 20647-20651). Although mRNA levels of BACE2 are low in the brain, it has been shown that the BACE2 protein (also known as Asp1) is present in brain as well as in neurons within the hippocampus, frontal cortex, and temporal cortex of the brain (Hussain, et. al., 2000. Molec. and Cell. Neuroscience 16, 609-619). Furthermore, it has also been shown that BACE2 can cleave APP at the β-secretase site (Hussain, et. al., 2000. Molec. And Cell. Neuroscience 16, 609-619; Farzan, et. al., 2000, Proc. Natl. Acad. Sci. 97, 9712-9717).

Although BACE2 can cleave APP at the β-secretase site, the predominant β-secretase in cells appears to be BACE. However, it has been shown that BACE2 also acts as an α-secretase, cleaving APP within the amyloid P domain to produce nonpathogenic soluble molecules (Farzan, et. al., 2000, Proc. Natl. Acad. Sci. 97, 9712-9717; Yan, et. al., 2001, J. Biol. Chem. 276, 34019-34027; Fluher, et. al., 2002, J. Neurochem. 81, 1011-1020).

In addition to its putative role in Alzheimer disease and Down syndrome, BACE2 has been implicated in tumor growth and metastasis in certain cancers such as breast and colon cancer (Xin, et. al., 2000, Biochimica et Biophysica Acta 1501, 125-137).

Several different inhibitors of BACE have been reported: P₁₀-P_{4′} StatVal inhibitor peptide (Sinha, et. al., 1999, Nature 402, 537-540) and OM99-1 and OM99-2 (Ghosh, et. al., 2000, J. Am. Chem. Soc. 122, 3522-3523). P₁₀-P_{4′} StatVal and OM99-2 have also been shown to be effective inhibitors of BACE2 (Gruniger-Leitch, et. al., 2002, Am. J. biol. Chem. 277, 4687-4693).

Modulating BACE2 activity within a cell could have therapeutic value in the treatment of Alzheimer disease, Down syndrome, and various cancers. Inhibiting the activity of BACE2 as a β-secretase could prevent the formation of amyloid-βplaques (Hardy, J. and Selkoe, D., 2002, Science 297, 353-356). Conversely, increased levels of BACE2 in cells, acting as an α-secretase, may compete with BACE, resulting in the formation of more soluble, nonpathogenic molecules and less insoluble amyloid-α (Fluher, et. al., 2002, J. Neurochem. 81, 1011-1020). In addition, inhibition of BACE2 may interfere with tumor growth or the metastatic pathway (Xin, et. al., 2000, Biochimica et Biophysica Acta 1501, 125-137). Given the potential value of BACE2 as a drug target, there is a need in the art for compounds that selectively bind to isoforms of human BACE2. The present invention is directed towards two novel BACE2 isoforms (BACE2sv1 and BACE2sv2) and uses thereof.

### SUMMARY OF THE INVENTION

Microarray experiments and RT-PCR have been used to identify and confirm the presence of novel splice variants of human BACE2 mRNA. More specifically, the present invention features polynucleotides encoding different protein isoforms of BACE2. A polynucleotide sequence encoding BACE2sv1 is provided by SEQ ID NO 1. An amino acid sequence for BACE2sv1 is provided by SEQ ID NO 2. A polynucleotide sequence encoding BACE2sv2 is provided by SEQ ID NO 3. An amino acid sequence for BACE2sv2 is provided by SEQ ID NO 4.

Thus, a first aspect of the present invention describes a purified BACE2sv1 encoding nucleic acid and a purified BACE2sv2 encoding nucleic acid. The BACE2sv1 encoding nucleic acid comprises SEQ ID NO 1 or the complement thereof. The BACE2sv2 encoding nucleic acid comprises SEQ ID NO 3 or the complement thereof. Reference to the presence of one region does not indicate that another region is not present. For example, in different embodiments the inventive nucleic acid can comprise, consist, or consist essentially of an encoding nucleic acid sequence of SEQ ID NO 1, or can comprise, consist, or consist essentially of the nucleic acid sequence of SEQ ID NO 3.

Another aspect of the present invention describes a purified BACE2sv1 polypeptide that can comprise, consist or consist essentially of the amino acid sequence of SEQ ID NO 2. An additional aspect describes a purified BACE2sv2 polypeptide that can comprise, consist, or consist essentially of the amino acid sequence of SEQ ID NO 4.

Another aspect of the present invention describes expression vectors. In one embodiment of the invention, the inventive expression vector comprises a nucleotide sequence encoding a polypeptide comprising, consisting, or consisting essentially of SEQ ID NO 2, wherein the nucleotide sequence is transcriptionally coupled to an exogenous promoter. In another embodiment, the inventive expression vector comprises a nucleotide sequence encoding a polypeptide comprising, consisting, or consisting essentially of SEQ ID NO 4, wherein the nucleotide sequence is transcriptionally coupled to an exogenous promoter.

Alternatively, the nucleotide sequence comprises, consists, or consists essentially of SEQ ID NO 1, and is transcriptionally coupled to an exogenous promoter. In another embodiment, the nucleotide sequence comprises, consists, or consists essentially of SEQ ID NO 3, and is transcriptionally coupled to an exogenous promoter.

Another aspect of the present invention describes recombinant cells comprising expression vectors comprising, consisting, or consisting essentially of the above-described sequences and the promoter is recognized by an RNA polymerase present in the cell. Another aspect of the present invention describes a recombinant cell made by a process comprising the step of introducing into the cell an expression vector comprising a nucleotide sequence comprising, consisting, or consisting essentially of SEQ ID NO 1, SEQ ID NO 3, or a nucleotide sequence encoding a polypeptide comprising, consisting, or consisting essentially of an amino acid sequence of SEQ ID NO 2 or SEQ ID NO 4, wherein the nucleotide sequence is transcriptionally coupled to an exogenous promoter. The expression vector can be used to insert recombinant nucleic acid into the host genome or can exist as an autonomous piece of nucleic acid.

Another aspect of the present invention describes a method of producing BACE2sv1 orBACE2sv2 polypeptide comprising SEQ ID NO 2 or SEQ ID NO 4, respectively. The method involves the step of growing a recombinant cell containing an inventive expression vector under conditions wherein the polypeptide is expressed from the expression vector.

Another aspect of the present invention features a purified antibody preparation comprising an antibody that binds selectively to BACE2sv1 as compared to one or more BACE2 isoform polypeptides that are not BACE2sv1. In another embodiment, a purified antibody preparation is provided comprising antibody that binds selectively to BACE2sv2 as compared to one or more BACE2 isoform polypeptides that are not BACE2sv2.

Another aspect of the present invention provides a method of screening for a compound that binds to BACE2sv1, BACE2sv2, or fragments thereof. In one embodiment, the method comprises the steps of: (a) expressing a polypeptide comprising the amino acid sequence of SEQ ID NO 2 or a fragment thereof from recombinant nucleic acid; (b) providing to said polypeptide a labeled BACE2 ligand that binds to said polypeptide and a test preparation comprising one or more test compounds; (c) and measuring the effect of said test preparation on binding of said test preparation to said polypeptide comprising SEQ ID NO 2. Alternatively, this method could be performed using SEQ ID NO 4 in place of SEQ ID NO 2.

In another embodiment of the method, a compound is identified that binds selectively to BACE2sv1 polypeptide as compared to one or more BACE2 isoform polypeptides that are not BACE2sv1. This method comprises the steps of: providing a BACE2sv1 polypeptide comprising SEQ ID NO 2; providing a BACE2 isoform polypeptide that is not BACE2sv1, contacting said BACE2sv1 polypeptide and said BACE2 isoform polypeptide that is not BACE2sv1 with a test preparation comprising one or more test compounds; and determining the binding of said test preparation to said BACE2sv1 polypeptide and to BACE2 isoform polypeptide that is not BACE2sv1, wherein a test preparation that binds to said BACE2sv1 polypeptide but does not bind to said BACE2 isoform polypeptide that is not BACE2sv1 contains a compound that selectively binds said BACE2sv1 polypeptide. Alternatively, the same method can be performed using BACE2sv2 polypeptide comprising, consisting, or consisting essentially of SEQ ID NO 4.

In another embodiment of the invention, a method is provided for screening for a compound able to bind to or interact with a BACE2sv1 protein or a fragment thereof comprising the steps of: expressing a BACE2sv1 polypeptide comprising SEQ ID NO 2 or a fragment thereof from a recombinant nucleic acid; providing to said polypeptide a labeled BACE2 ligand that binds to said polypeptide and a test preparation comprising one or more compounds; and measuring the effect of said test preparation on binding of said labeled BACE2 ligand to said polypeptide, wherein a test preparation that alters the binding of said labeled BACE2 ligand to said polypeptide contains a compound that binds to or interacts with said polypeptide. In an alternative embodiment, the method is performed using BACE2sv2 polypeptide comprising, consisting, or consisting essentially of SEQ ID NO 4 or a fragment thereof.

Other features and advantages of the present invention are apparent from the additional descriptions provided herein including the different examples. The provided examples illustrate different components and methodology useful in practicing the present invention. The examples do not limit the claimed invention. Based on the present disclosure the skilled artisan can identify and employ other components and methodology useful for practicing the present invention.

### BRIEF DESCRIPTION OF THE FIGURES

FIG. 1A illustrates the exon structure of BACE2 mRNA corresponding to the known reference form of BACE2 mRNA (labeled NM_{—}012105). FIG. 1B illustrates one of the inventive short form splice variants of BACE2 mRNA (labeled BACE2sv2). The small arrows above exons 6 and 9 show the positions of the oligonucleotide primers used to perform RT-PCR assays to confirm the exon structure of BACE2 mRNA in 44 human tissue and cell line samples. The nucleotide sequences shown in boxes below the exon structure diagrams of the BACE2 and BACE2sv2 mRNAs depict the nucleotide sequences of the exon junctions resulting from the splicing of exon 6 to exon 7, and exon 8 to exon 9 in the case of the BACE2 mRNA (FIG. 1A); and the splicing of exon 6 to exon 9 in the case of BACE2sv2 mRNA (FIG. 1B). In FIG. 1A, the nucleotides shown in italics represent the 20 nucleotides at the 3′ end of exon 6 and the nucleotides shown in underline represent the 20 nucleotides at the 5′ end of exon 9. In FIG. 1B, nucleotides in italics associated with the exon 6 to exon 9 splice junction represent the 20 nucleotides at the 3′ end of exon 6, while the nucleotides in underline associated with the exon 6 to exon 9 splice junction represent the 20 nucleotides at the 5′ end of exon 9.

### DEFINITIONS

Unless defined otherwise, all technical and scientific terms used herein have the meaning commonly understood by one of ordinary skill in the art to which this invention belongs.

As used herein, “BACE2” refers to an aspartyl protease protein (NP_{—}036237). In contrast, reference to a BACE2 isoform, includes NP_{—}036237 and other polypeptide isoform variants of BACE2.

As used herein, “BACE2sv1” and “BACE2sv2” refer to splice variant isoforms of human BACE2 protein, wherein the splice variants have the amino acid sequence set forth in SEQ ID NO 2 (for BACE2sv1) and SEQ ID NO 4 (for BACE2sv2).

As used herein, “BACE2” refers to polynucleotides encoding BACE2.

As used herein, “BACE2sv1” refers to polynucleotides encoding BACE2sv1 having an amino acid sequence set forth in SEQ ID NO 2. As used herein, “BACE2sv2” refers to polynucleotides encoding BACE2sv2 having an amino acid sequence set forth in SEQ ID NO 4.

As used herein, an “isolated nucleic acid” is a nucleic acid molecule that exists in a physical form that is nonidentical to any nucleic acid molecule of identical sequence as found in nature; “isolated”does not require, although it does not prohibit, that the nucleic acid so described has itself been physically removed from its native environment. For example, a nucleic acid can be said to be “isolated” when it includes nucleotides and/or internucleoside bonds not found in nature. When instead composed of natural nucleosides in phosphodiester linkage, a nucleic acid can be said to be “isolated” when it exists at a purity not found in nature, where purity can be adjudged with respect to the presence of nucleic acids of other sequence, with respect to the presence of proteins, with respect to the presence of lipids, or with respect the presence of any other component of a biological cell, or when the nucleic acid lacks sequence that flanks an otherwise identical sequence in an organism's genome, or when the nucleic acid possesses sequence not identically present in nature. As so defined, “isolated nucleic acid” includes nucleic acids integrated into a host cell chromosome at a heterologous site, recombinant fusions of a native fragment to a heterologous sequence, recombinant vectors present as episomes or as integrated into a host cell chromosome.

A “purified nucleic acid” represents at least 10% of the total nucleic acid present in a sample or preparation. In preferred embodiments, the purified nucleic acid represents at least about 50%, at least about 75%, or at least about 95% of the total nucleic acid in a isolated nucleic acid sample or preparation. Reference to “purified nucleic acid” does not require that the nucleic acid has undergone any purification and may include, for example, chemically synthesized nucleic acid that has not been purified.

The phrases “isolated protein”, “isolated polypeptide”, “isolated peptide” and “isolated oligopeptide” refer to a protein (or respectively to a polypeptide, peptide, or oligopeptide) that is nonidentical to any protein molecule of identical amino acid sequence as found in nature; “isolated” does not require, although it does not prohibit, that the protein so described has itself been physically removed from its native environment. For example, a protein can be said to be “isolated” when it includes amino acid analogues or derivatives not found in nature, or includes linkages other than standard peptide bonds. When instead composed entirely of natural amino acids linked by peptide bonds, a protein can be said to be “isolated” when it exists at a purity not found in nature—where purity can be adjudged with respect to the presence of proteins of other sequence, with respect to the presence of non-protein compounds, such as nucleic acids, lipids, or other components of a biological cell, or when it exists in a composition not found in nature, such as in a host cell that does not naturally express that protein.

As used herein, a “purified polypeptide” (equally, a purified protein, peptide, or oligopeptide) represents at least 10% of the total protein present in a sample or preparation, as measured on a weight basis with respect to total protein in a composition. In preferred embodiments, the purified polypeptide represents at least about 50%, at least about 75%, or at least about 95% of the total protein in a sample or preparation. A “substantially purified protein” (equally, a substantially purified polypeptide, peptide, or oligopeptide) is an isolated protein, as above described, present at a concentration of at least 70%, as measured on a weight basis with respect to total protein in a composition. Reference to “purified polypeptide” does not require that the polypeptide has undergone any purification and may include, for example, chemically synthesized polypeptide that has not been purified.

As used herein, the term “antibody” refers to a polypeptide, at least a portion of which is encoded by at least one immunoglobulin gene, or fragment thereof, and that can bind specifically to a desired target molecule. The term includes naturally-occurring forms, as well as fragments and derivatives. Fragments within the scope of the term “antibody” include those produced by digestion with various proteases, those produced by chemical cleavage and/or chemical dissociation, and those produced recombinantly, so long as the fragment remains capable of specific binding to a target molecule. Among such fragments are Fab, Fab′, Fv, F(ab)′₂, and single chain Fv (scFv) fragments. Derivatives within the scope of the term include antibodies (or fragments thereof) that have been modified in sequence, but remain capable of specific binding to a target molecule, including: interspecies chimeric and humanized antibodies; antibody fusions; heteromeric antibody complexes and antibody fusions, such as diabodies (bispecific antibodies), single-chain diabodies, and intrabodies (see, e.g., Marasco (ed.), Intracellular Antibodies: Research and Disease Applications, Springer-Verlag New York, Inc. (1998) (ISBN: 3540641513). As used herein, antibodies can be produced by any known technique, including harvest from cell culture of native B lymphocytes, harvest from culture of hybridomas, recombinant expression systems, and phage display.

As used herein, a “purified antibody preparation” is a preparation where at least 10% of the antibodies present bind to the target ligand. In preferred embodiments, antibodies binding to the target ligand represent at least about 50%, at least about 75%, or at least about 95% of the total antibodies present. Reference to “purified antibody preparation” does not require that the antibodies in the preparation have undergone any purification.

As used herein, “specific binding” refers to the ability of two molecular species concurrently present in a heterogeneous (inhomogeneous) sample to bind to one another in preference to binding to other molecular species in the sample. Typically, a specific binding interaction will discriminate over adventitious binding interactions in the reaction by at least two-fold, more typically by at least 10-fold, often at least 100-fold; when used to detect analyte, specific binding is sufficiently discriminatory when determinative of the presence of the analyte in a heterogeneous (inhomogeneous) sample. Typically, the affinity or avidity of a specific binding reaction is least about 1 μM.

The term “antisense”, as used herein, refers to a nucleic acid molecule sufficiently complementary in sequence, and sufficiently long in that complementary sequence, as to hybridize under intracellular conditions to (i) a target mRNA transcript or (ii) the genomic DNA strand complementary to that transcribed to produce the target mRNA transcript.

The term “subject”, as used herein refers to an organism and to cells or tissues derived therefrom. For example the organism may be an animal, including but not limited to animals such as cows, pigs, horses, chickens, cats, dogs, etc., and is usually a mammal, and most commonly human.

### DETAILED DESCRIPTION OF THE INVENTION

This section presents a detailed description of the present invention and its applications. This description is by way of several exemplary illustrations, in increasing detail and specificity, of the general methods of this invention. These examples are non-limiting, and related variants that will be apparent to one of skill in the art are intended to be encompassed by the appended claims.

The present invention relates to the nucleic acid sequences encoding human BACE2sv1 and BACE2sv2 that are alternatively spliced isoforms of BACE2, and to the amino acid sequences encoding these proteins. SEQ ID NO 1 and SEQ ID NO 3 are polynucleotide sequences representing exemplary open reading frames that encode the BACE2sv1 and BACE2sv2 proteins, respectively. SEQ ID NO 2 shows the polypeptide sequence of BACE2sv1. SEQ ID NO 4 shows the polypeptide sequence of BACE2sv2.

BACE2sv1 and BACE2sv2 polynucleotide sequences encoding BACE2sv1 and BACE2sv2 proteins, as exemplified and enabled herein, include a number of specific, substantial and credible utilities. For example, BACE2sv1 and BACE2sv2 encoding nucleic acids were identified in an mRNA sample obtained from a human source (see Example 1). Such nucleic acids can be used as hybridization probes to distinguish between cells that produce BACE2sv1 and BACE2sv2 transcripts from human or non-human cells (including bacteria) that do not produce such transcripts. Similarly, antibodies specific for BACE2sv1 or BACE2sv2 can be used to distinguish between cells that express BACE2sv1 orBACE2sv2 from human or non-human cells (including bacteria) that do not express BACE2sv1 or BACE2sv2.

BACE2 may be an important drug target for the treatment of Alzheimer disease, Down syndrome, and/or certain cancers (Hardy, J. and Selkoe, D., 2002, Science 297, 353-356; Fluher, et. al., 2002, J. Neurochem. 81, 1011-1020; Xin, et. al., 2000, Biochimica et Biophysica Acta 1501, 125-137). Given the potential importance of BACE2 activity to the therapeutic management of these diseases, it is of value to identify BACE2 isoforms and identify BACE2-ligand compounds that are isoform specific, as well as compounds that are effective ligands for two or more different BACE2 isoforms. In particular, it may be important to identify compounds that are effective inhibitors of a specific BACE2 isoform activity, yet does not bind to or interact with a plurality of different BACE2 isoforms. Compounds that bind to or interact with multiple BACE2 isoforms may require higher drug doses to saturate multiple BACE2-isoform binding sites and thereby result in a greater likelihood of secondary non-therapeutic side effects. Furthermore, biological effects could also be caused by the interactions of a drug with the BACE2sv1 or BACE2sv2 isoforms specifically. For the foregoing reasons, BACE2sv1 and BACE2sv2 proteins represent useful compound binding targets and have utility in the identification of new BACE2-ligands exhibiting a preferred specificity profile and having greater efficacy for their intended use.

In some embodiments, BACE2sv1 and BACE2sv2 activity is modulated by a ligand compound to achieve one or more of the following: prevent or reduce the risk of occurrence of Alzheimer disease; prevent or reduce the risk of occurrence of certain cancers (in particular breast and colon cancer); or prevent the formation of amyloid-β plaques in persons with Down syndrome.

Compounds modulating BACE2sv1 or BACE2sv2 include agonists, antagonists, and allosteric modulators. While not wishing to be limited to any particular theory of therapeutic efficacy, generally, but not always, BACE2sv1 or BACE2sv2 compounds may be used to inhibit aspartyl protease activity. Inhibitors of BACE2 may achieve clinical efficacy by a number of known or unknown mechanisms. In the case of cancer treatment it is hypothesized that inhibition of BACE2 may interfere with tumor growth or the metastatic pathway (Xin, et. al., 2000, Biochimica et Biophysica Acta 1501, 125-137). In the case of treatment of Alzheimer disease or Down syndrome, inhibiting the activity of BACE2 as a β-secretase may prevent the formation of amyloid-β plaques (Hardy, J. and Selkoe, D., 2002, Science 297, 353-356).

BACE2sv1 or BACE2sv2 activity may also be affected by modulating the cellular abundance of transcripts encoding BACE2sv1 or BACE2sv2, respectively. Compounds modulating the abundance of transcripts encoding BACE2sv1 or BACE2sv2 include a cloned polynucleotide encoding BACE2sv1 or BACE2sv2, respectively, that can express BACE2sv2 or BACE2sv2 in vivo, antisense nucleic acids targeted to BACE2sv1 or BACE2sv2 transcripts, and enzymatic nucleic acids, such as ribozymes and RNAi, targeted to BACE2sv1 or BACE2sv2 transcripts.

In some embodiments, BACE2sv1 or BACE2sv2 activity is modulated to achieve a therapeutic effect upon diseases in which regulation of aspartyl protease activity is desirable. For example, Alzheimer disease or Down syndrome may be treated by modulating BACE2sv1 or BACE2sv2 activities to increase cleavage of APP within the amyloid-β domain, thus forming soluble nonpathogenic molecules and reducing the amount of APP available to β-secretase (BACE) and thus the formation of insoluble amyloid-β.

### BACE2sv1 or BACE2sv2 Nucleic Acid

BACE2sv1 nucleic acids contain regions that encode for polypeptides comprising, consisting, or consisting essentially of SEQ ID NO 2. BACE2sv2 nucleic acids contain regions that encode for polypeptides comprising, consisting, or consisting essentially of SEQ ID NO 4. The BACE2sv1 and BACE2sv2 nucleic acids have a variety of uses, such as use as a hybridization probe or PCR primer to identify the presence of BACE2sv1 or BACE2sv2 nucleic acids, respectively; use as a hybridization probe or PCR primer to identify nucleic acids encoding for proteins related to BACE2sv1 or BACE2sv2, respectively; and/or use for recombinant expression of BACE2sv1 or BACE2sv2 polypeptides, respectively. In particular, BACE2sv1 polynucleotides do not have the polynucleotide regions that comprise exons 1 and 2 of the BACE2 gene, but instead have an alternative 5′ exon. BACE2sv2 polynucleotides do not have the polynucleotide regions that comprise exons 7 and 8 of the BACE2 gene.

Regions in BACE2sv1 or BACE2sv2 nucleic acid that do not encode for BACE2sv1 or BACE2sv2, or are not found in SEQ ID NO 1 or SEQ ID NO 3, if present, are preferably chosen to achieve a particular purpose. Examples of additional regions that can be used to achieve a particular purpose include: a stop codon that is effective at protein synthesis termination; capture regions that can be used as part of an ELISA sandwich assay; reporter regions that can be probed to indicate the presence of the nucleic acid; expression vector regions; and regions encoding for other polypeptides.

The guidance provided in the present application can be used to obtain the nucleic acid sequence encoding BACE2sv1 or BACE2sv2 related proteins from different sources. Obtaining nucleic acids encoding BACE2sv1 or BACE2sv2 related proteins from different sources is facilitated by using sets of degenerative probes and primers and the proper selection of hybridization conditions. Sets of degenerative probes and primers are produced taking into account the degeneracy of the genetic code. Adjusting hybridization conditions is useful for controlling probe or primer specificity to allow for hybridization to nucleic acids having similar sequences.

Techniques employed for hybridization detection and PCR cloning are well known in the art. Nucleic acid detection techniques are described, for example, in Sambrook, et al., in Molecular Cloning, A Laboratory Manual, 2^{nd} Edition, Cold Spring Harbor Laboratory Press, 1989. PCR cloning techniques are described, for example, in White, Methods in Molecular Cloning, volume 67, Humana Press, 1997.

BACE2sv1 or BACE2sv2 probes and primers can be used to screen nucleic acid libraries containing, for example, cDNA. Such libraries are commercially available, and can be produced using techniques such as those described in Ausubel, Current Protocols in Molecular Biology, John Wiley, 1987-1998.

Starting with a particular amino acid sequence and the known degeneracy of the genetic code, a large number of different encoding nucleic acid sequences can be obtained. The degeneracy of the genetic code arises because almost all amino acids are encoded for by different combinations of nucleotide triplets or “codons”. The translation of a particular codon into a particular amino acid is well known in the art (see, e.g., Lewin GENES IV, p. 119, Oxford University Press, 1990). Amino acids are encoded for by codons as follows:

A=Ala=Alanine: codons GCA, GCC, GCG, GCU

C=Cys=Cysteine: codons UGC, UGU

D=Asp=Aspartic acid: codons GAC, GAU

E=Glu=Glutamic acid: codons GAA, GAG

F=Phe=Phenylalanine: codons UUC, UUU

G=Gly=Glycine: codons GGA, GGC, GGG, GGU

H=His=Histidine: codons CAC, CAU

I=Ile=Isoleucine: codons AUA, AUC, AUU

K=Lys=Lysine: codons AAA, AAG

L=Leu=Leucine: codons UUA, UUG, CUA, CUC, CUG, CUU

M=Met=Methionine: codon AUG

N=Asn=Asparagine: codons AAC, AAU

P=Pro=Proline: codons CCA, CCC, CCG, CCU

Q=Gln=Glutamine: codons CAA, CAG

R=Arg=Arginine: codons AGA, AGG, CGA, CGC, CGG, CGU

S=Ser=Serine: codons AGC, AGU, UCA, UCC, UCG, UCU

T=Thr=Threonine: codons ACA, ACC, ACG, ACU

V=Val=Valine: codons GUA, GUC, GUG, GUU

W=Trp=Tryptophan: codon UGG

Y=Tyr=Tyrosine: codons UAC, UAU

Nucleic acid having a desired sequence can be synthesized using chemical and biochemical techniques. Examples of chemical techniques are described in Ausubel, Current Protocols in Molecular Biology, John Wiley, 1987-1998, and Sambrook et al., in Molecular Cloning, A Laboratory Manual, 2^{nd} Edition, Cold Spring Harbor Laboratory Press, 1989. In addition, long polynucleotides of a specified nucleotide sequence can be ordered from commercial vendors, such as Blue Heron Biotechnology, Inc. (Bothell, Wash.).

Biochemical synthesis techniques involve the use of a nucleic acid template and appropriate enzymes such as DNA and/or RNA polymerases. Examples of such techniques include in vitro amplification techniques such as PCR and transcription based amplification, and in vivo nucleic acid replication. Examples of suitable techniques are provided by Ausubel, Current Protocols in Molecular Biology, John Wiley, 1987-1998, Sambrook et al., in Molecular Cloning, A Laboratory Manual, 2^{nd} Edition, Cold Spring Harbor Laboratory Press, 1989, and U.S. Pat. No. 5,480,784.

### BACE2sv2 Probes

Probes for BACE2sv2 contain a region that can specifically hybridize to BACE2sv2 target nucleic acids, under appropriate hybridization conditions, and can distinguish BACE2sv2 nucleic acids from non-target nucleic acids, in particular BACE2 polynucleotides containing exons 7 and 8. Probes for BACE2sv2 can also contain nucleic acid regions that are not complementary with BACE2sv2 nucleic acids.

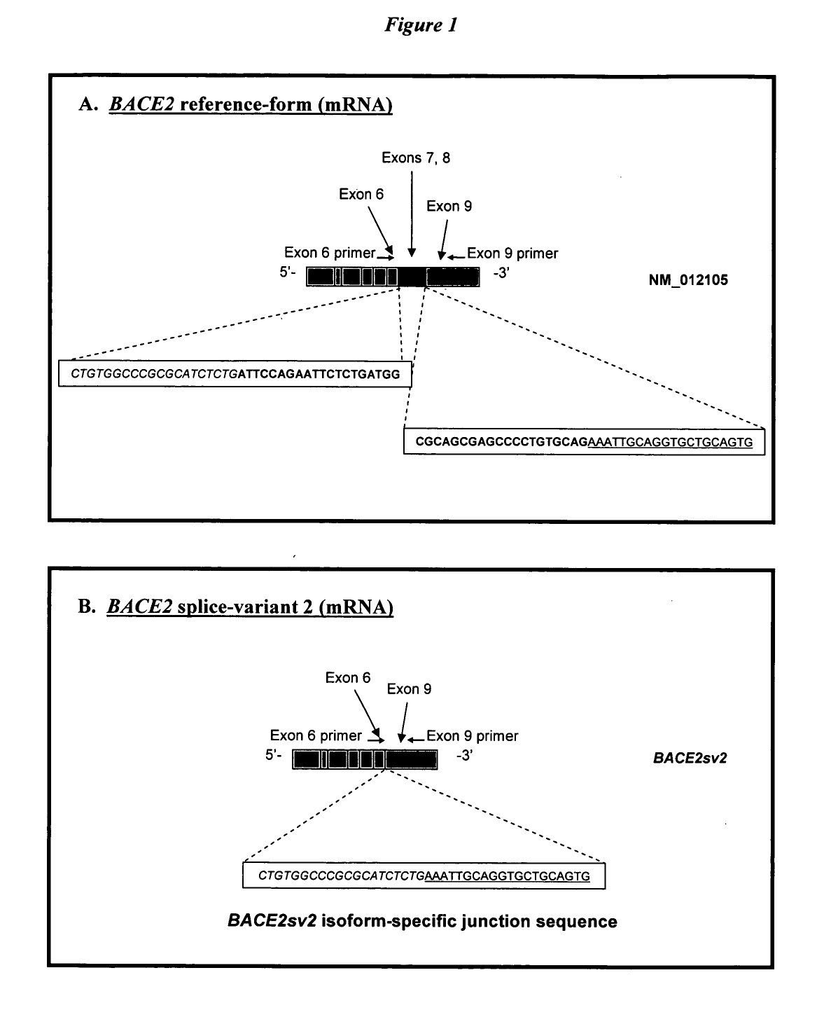

In embodiments where, for example, BACE2sv2 polynucleotide probes are used in hybridization assays to specifically detect the presence of BACE2sv2 polynucleotides in samples, the BACE2sv2 polynucleotides comprise at least 20 nucleotides of the BACE2sv2 sequence that correspond to the novel exon junction polynucleotide region. In particular, for detection of BACE2sv2, the probe comprises at least 20 nucleotides of the BACE2sv2 sequence that corresponds to an exon junction polynucleotide created by the alternative splicing of exon 6 to exon 9 of the primary transcript of the BACE2 gene (see FIG. 1A and B). For example, the polynucleotide sequence: 5′ CGCATCTCTGAAATTGCAGG 3′ [SEQ ID NO 5] represents one embodiment of such an inventive BACE2sv2 polynucleotide wherein a first 10 nucleotides region is complementary and hybridizable to the 3′ end of exon 6 of the BACE2 gene and a second 10 nucleotide region is complementary and hybridizable to the 5′ end of exon 9 of the BACE2 gene (see FIG. 1B).

In some embodiments, the first 20 nucleotides of a BACE2sv2 probe comprise a first continuous region of 5 to 15 nucleotides that is complementary and hybridizable to the 3′ end of exon 6 and a second continuous region of 5 to 15 nucleotides that is complementary and hybridizable to the 5′ end of exon 9.

In other embodiments, the BACE2sv2 polynucleotide comprises at least 40, 60, 80 or 100 nucleotides of the BACE2sv2 sequence that correspond to a junction polynucleotide region created by the alternative splicing of exon 6 to exon 9 of the primary transcript of the BACE2 gene. In embodiments involving BACE2sv2, the BACE2sv2 polynucleotide is selected to comprise a first continuous region of at least 5 to 15 nucleotides that is complementary and hybridizable to the 3′ end of exon 6 and a second continuous region of at least 5 to 15 nucleotides that is complementary and hybridizable to the 5′ end of exon 9. As will be apparent to a person of skill in the art, a large number of different polynucleotide sequences from the region of the exon 6 to exon 9 splice junction may be selected which will, under appropriate hybridization conditions, have the capacity to detectably hybridize to BACE2sv2 polynucleotides, and yet will hybridize to a much less extent or not at all to BACE2 isoform polynucleotides wherein exon 6 is not spliced to exon 9.

Preferably, non-complementary nucleic acid that is present has a particular purpose such as being a reporter sequence or being a capture sequence. However, additional nucleic acid need not have a particular purpose as long as the additional nucleic acid does not prevent the BACE2sv2 nucleic acid from distinguishing between target polynucleotides, e.g., BACEsv2 polynucleotides, and non-target polynucleotides, including, but not limited to BACE2 polynucleotides not comprising the exon 6 to exon 9 splice junction.

Hybridization occurs through complementary nucleotide bases. Hybridization conditions determine whether two molecules, or regions, have sufficiently strong interactions with each other to form a stable hybrid.

The degree of interaction between two molecules that hybridize together is reflected by the melting temperature (Tₘ) of the produced hybrid. The higher the Tₘ the stronger the interactions and the more stable the hybrid. Tₘ is effected by different factors well known in the art such as the degree of complementarity, the type of complementary bases present (e.g., A-T hybridization versus G-C hybridization), the presence of modified nucleic acid, and solution components (e.g., Sambrook, et al., in Molecular Cloning, A Laboratory Manual, 2^{nd} Edition, Cold Spring Harbor Laboratory Press, 1989).

Stable hybrids are formed when the Tₘ of a hybrid is greater than the temperature employed under a particular set of hybridization assay conditions. The degree of specificity of a probe can be varied by adjusting the hybridization stringency conditions. Detecting probe hybridization is facilitated through the use of a detectable label. Examples of detectable labels include luminescent, enzymatic, and radioactive labels.

Examples of stringency conditions are provided in Sambrook, et al., in Molecular Cloning, A Laboratory Manual, 2^{nd} Edition, Cold Spring Harbor Laboratory Press, 1989. An example of high stringency conditions is as follows: Prehybridization of filters containing DNA is carried out for 2 hours to overnight at 65° C. in buffer composed of 6×SSC, 5×Denhardt's solution, and 100 μg/ml denatured salmon sperm DNA. Filters are hybridized for 12 to 48 hours at 65° C. in prehybridization mixture containing 100 μg/ml denatured salmon sperm DNA and 5-20×10⁶ cpm of ³²P-labeled probe. Filter washing is done at 37° C. for 1 hour in a solution containing 2×SSC, 0.1% SDS. This is followed by a wash in 0.1×SSC, 0.1% SDS at 50° C. for 45 minutes before autoradiography. Other procedures using conditions of high stringency would include, for example, either a hybridization step carried out in 5×SSC, 5×Denhardt's solution, 50% formamide at 42° C. for 12 to 48 hours or a washing step carried out in 0.2×SSPE, 0.2% SDS at 65° C. for 30 to 60 minutes.

### Recombinant Expression

BACE2sv1 or BACE2sv2 polynucleotides, such as those comprising SEQ ID NO 1 or SEQ ID NO 3, respectively, can be used to make BACE2sv1 or BACE2sv2 polypeptides, respectively. In particular, BACE2sv1 or BACE2sv2 polypeptides can be expressed from recombinant nucleic acids in a suitable host or in vitro using a translation system. Recombinantly expressed BACE2sv1 or BACE2sv2 polypeptides can be used, for example, in assays to screen for compounds that bind BACE2sv1 or BACE2sv2, respectively. Alternatively, BACE2sv1 or BACE2sv2 polypeptides can also be used to screen for compounds that bind to one or more BACE2 isoforms but do not bind to BACE2sv1 or BACE2sv2, respectively.

In some embodiments, expression is achieved in a host cell using an expression vector. An expression vector contains recombinant nucleic acid encoding a polypeptide along with regulatory elements for proper transcription and processing. The regulatory elements that may be present include those naturally associated with the recombinant nucleic acid and exogenous regulatory elements not naturally associated with the recombinant nucleic acid. Exogenous regulatory elements such as an exogenous promoter can be useful for expressing recombinant nucleic acid in a particular host.

Generally, the regulatory elements that are present in an expression vector include a transcriptional promoter, a ribosome binding site, a terminator, and an optionally present operator. Another preferred element is a polyadenylation signal providing for processing in eukaryotic cells. Preferably, an expression vector also contains an origin of replication for autonomous replication in a host cell, a selectable marker, a limited number of useful restriction enzyme sites, and a potential for high copy number. Examples of expression vectors are cloning vectors, modified cloning vectors, and specifically designed plasmids and viruses.

Expression vectors providing suitable levels of polypeptide expression in different hosts are well known in the art. Mammalian expression vectors well known in the art include, but are not restricted to, pcDNA3 (Invitrogen, Carlsbad Calif.), pSecTag2 (Invitrogen), pMC1 neo (Stratagene, La Jolla Calif.), pXT1 Stratagene), pSG5 (Stratagene), pCMVLac1 (Stratagene), pCI-neo (Promega), EBO-pSV2-neo (ATCC 37593), pBPV-1(8-2) (ATCC 37110), pdBPV-MMTneo(342-12) (ATCC 37224), pRSVgpt (ATCC 37199), pRSVneo (ATCC 37198), pSV2-dhfr (ATCC 37146) and pUCTag (ATCC 37460), and. Bacterial expression vectors well known in the art include pET11a (Novagen), pBluescript SK (Stratagene, La Jolla), pQE-9 (Qiagen Inc., Valencia), lambda gt11 (Invitrogen), pcDNAII (Invitrogen), and pKK223-3 (Pharmacia). Funga1 cell expression vectors well known in the art include pPICZ (Invitrogen) and pYES2 (Invitrogen), Pichia expression vector (Invitrogen). Insect cell expression vectors well known in the art include Blue Bac III (Invitrogen), pBacPAK8 (CLONTECH, Inc., Palo Alto) and PfastBacHT (Invitrogen, Carlsbad).

Recombinant host cells may be prokaryotic or eukaryotic. Examples of recombinant host cells include the following: bacteria such as E. coli; fungal cells such as yeast; mammalian cells such as human, bovine, porcine, monkey and rodent; and insect cells such as Drosophila and silkworm derived cell lines. Commercially available mammalian cell lines include L cells L-M(TK⁻) (ATCC CCL 1.3), L cells L-M (ATCC CCL 1.2), 293 (ATCC CRL 1573), Raji (ATCC CCL 86), CV-1 (ATCC CCL 70), COS-1 (ATCC CRL 1650), COS-7 (ATCC CRL 1651), CHO-K1 (ATCC CCL 61), 3T3 (ATCC CCL 92), NIH/3T3 (ATCC CRL 1658), HeLa (ATCC CCL 2), C127I (ATCC CRL 1616), BS-C-1 (ATCC CCL 26) MRC-5 (ATCC CCL 171), and HEK 293 cells (ATCC CRL-1573).

To enhance expression in a particular host it may be useful to modify the sequence provided in SEQ ID NO 1 or SEQ ID NO 3 to take into account codon usage of the host. Codon usages of different organisms are well known in the art (see, Ausubel, Current Protocols in Molecular Biology, John Wiley, 1987-1998, Supplement 33 Appendix 1C).

Expression vectors may be introduced into host cells using standard techniques. Examples of such techniques include transformation, transfection, lipofection, protoplast fusion, and electroporation.

Nucleic acids encoding for a polypeptide can be expressed in a cell without the use of an expression vector employing, for example, synthetic mRNA or native mRNA. Additionally, mRNA can be translated in various cell-free systems such as wheat germ extracts and reticulocyte extracts, as well as in cell based systems, such as frog oocytes. Introduction of mRNA into cell based systems can be achieved, for example, by microinjection or electroporation.

### BACE2sv1 and BACE2sv2 Polypeptide

BACE2sv1 polypeptides contain an amino acid sequence comprising, consisting or consisting essentially of SEQ ID NO 2. BACE2sv2 polypeptides contain an amino acid sequence comprising, consisting or consisting essentially of SEQ ID NO 4. BACE2sv1 or BACE2sv2 polypeptides have a variety of uses, such as providing a marker for the presence of BACE2sv1 or BACE2sv2, respectively; use as an immunogen to produce antibodies binding to BACE2sv1 or BACE2sv2, respectively; use as a target to identify compounds binding selectively to BACE2sv1 or BACE2sv2, respectively; or use in an assay to identify compounds that bind to one or more iosforms of BACE2 but do not bind to or interact with BACE2sv1 or BACE2sv2, respectively.

In chimeric polypeptides containing one or more regions from BACE2sv1 or BACE2sv2 and one or more regions not from BACE2sv1 or BACE2sv2, respectively, the region(s) not from BACE2sv1 or BACE2sv2, respectively, can be used, for example, to achieve a particular purpose or to produce a polypeptide that can substitute for BACE2sv1 or BACE2sv2, or fragments thereof. Particular purposes that can be achieved using chimeric BACE2sv1 or BACE2sv2 polypeptides include providing a marker for BACE2sv1 or BACE2sv2 activity, respectively, enhancing an immune response, and modulating aspartyl protease activity or levels of BACE2.

Polypeptides can be produced using standard techniques including those involving chemical synthesis and those involving biochemical synthesis. Techniques for chemical synthesis of polypeptides are well known in the art (see e.g., Vincent, in Peptide and Protein Drug Delivery, New York, N.Y., Dekker, 1990).

Biochemical synthesis techniques for polypeptides are also well known in the art. Such techniques employ a nucleic acid template for polypeptide synthesis. The genetic code providing the sequences of nucleic acid triplets coding for particular amino acids is well known in the art (see, e.g., Lewin GENES IV, p. 119, Oxford University Press, 1990). Examples of techniques for introducing nucleic acid into a cell and expressing the nucleic acid to produce protein are provided in references such as Ausubel, Current Protocols in Molecular Biology, John Wiley, 1987-1998, and Sambrook, et al., in Molecular Cloning, A Laboratory Manual, 2^{nd} Edition, Cold Spring Harbor Laboratory Press, 1989.

### Functional BACE2sv1 and BACE2sv2

Functional BACE2sv1 or BACE2sv2 are different protein isoforms of BACE2. The identification of the amino acid and nucleic acid sequences of BACE2sv1 or BACE2sv2 provide tools for obtaining functional proteins related to BACE2sv1 or BACE2sv2, respectively, from other sources; for producing BACE2sv1 or BACE2sv2 chimeric proteins; and for producing functional derivatives of SEQ ID NO 2, or SEQ ID NO 4.

BACE2sv1 or BACE2sv2 polypeptides can be readily identified and obtained based on their sequence similarity to BACE2sv1 (SEQ ID NO 2), or BACE2sv2 (SEQ ID NO 4), respectively. In particular, BACE2sv1 polypeptides lack the amino acids encoded by the first 687 nucleotides of the coding sequence of the BACE2 gene. Initiation at a downstream AUG of a bicistronic RNA is a fairly common event and can be associated with disease (Meijer and Thomas, 2002 Biochem. J., 367:1-11; Kozak, 2002, Mammalian Genome 13:401-410). BACE2sv2 polypeptides lack the amino acids encoded by exons 7 and 8 of the BACE2 gene. The deletion of exons 7 and 8 and the splicing of exon 6 to exon 9 of the BACE2 hnRNA transcript results in a shift of the protein reading frame at the exon 6 to exon 9 splice junction, thereby creating a carboxy-terminal peptide region that is unique to the BACE2sv2 polypeptide as compared to other known BACE2 isoforms. The frame shift creates a premature termination codon fifty-four nucleotides downstream of the exon 6/exon 9 splice junction. Thus, BACE2sv2 polypeptides are lacking the amino acids encoded by the nucleotides downstream of the premature stop codon.

Both the amino acid and nucleic acid sequences of BACE2sv1 or BACE2sv2 can be used to help identify and obtain BACE2sv1 or BACE2sv2 polypeptides, respectively. For example, SEQ ID NO 1 can be used to produce degenerative nucleic acid probes or primers for identifying and cloning nucleic acid polynucleotides encoding for a BACE2sv1 polypeptide. In addition, polynucleotides comprising, consisting, or consisting essentially of SEQ ID NO 1 or fragments thereof, can be used under conditions of moderate stringency to identify and clone nucleic acids encoding BACE2sv1 polypeptides from a variety of different organisms. The same methods can also be performed with polynucleotides comprising, consisting, or consisting essentially of SEQ ID NO 3, or fragments thereof, to identify and clone nucleic acids encoding BACE2sv2.

The use of degenerative probes and moderate stringency conditions for cloning is well known in the art. Examples of such techniques are described by Ausubel, Current Protocols in Molecular Biology, John Wiley, 1987-1998, and Sambrook, et al., in Molecular Cloning, A Laboratory Manual, 2^{nd} Edition, Cold Spring Harbor Laboratory Press, 1989.

Starting with BACE2sv1 or BACE2sv2 obtained from a particular source, derivatives can be produced. Such derivatives include polypeptides with amino acid substitutions, additions and deletions. Changes to BACE2sv1 or BACE2sv2 to produce a derivative having essentially the same properties should be made in a manner not altering the tertiary structure of BACE2sv1 or BACE2sv2, respectively.

Differences in naturally occurring amino acids are due to different R groups. An R group affects different properties of the amino acid such as physical size, charge, and hydrophobicity. Amino acids are can be divided into different groups as follows: neutral and hydrophobic (alanine, valine, leucine, isoleucine, proline, tryptophan, phenylalanine, and methionine); neutral and polar (glycine, serine, threonine, tryosine, cysteine, asparagine, and glutamine); basic (lysine, arginine, and histidine); and acidic (aspartic acid and glutamic acid).

Generally, in substituting different amino acids it is preferable to exchange amino acids having similar properties. Substituting different amino acids within a particular group, such as substituting valine for leucine, arginine for lysine, and asparagine for glutamine are good candidates for not causing a change in polypeptide functioning.

Changes outside of different amino acid groups can also be made. Preferably, such changes are made taking into account the position of the amino acid to be substituted in the polypeptide. For example, arginine can substitute more freely for nonpolar amino acids in the interior of a polypeptide then glutamate because of its long aliphatic side chain (See, Ausubel, Current Protocols in Molecular Biology, John Wiley, 1987-1998, Supplement 33 Appendix 1C).

### BACE2sv1 and BACE2sv2 Antibodies

Antibodies recognizing BACE2sv1 or BACE2sv2 can be produced using a polypeptide containing SEQ ID NO 2 in the case of BACE2sv1, or SEQ ID NO 4 in the case of BACE2sv2, respectively, or a fragment thereof, as an immunogen. Preferably, a BACE2sv1 polypeptide used as an immunogen consists of a polypeptide of SEQ ID NO 2 or a SEQ ID NO 2 fragment having at least 10 contiguous amino acids in length corresponding to amino acids, including and downstream of, the amino terminal methionine of BACE2sv1. Preferably, a BACE2sv2 polypeptide used as an immunogen consists of a polypeptide of SEQ ID NO 4 or a SEQ ID NO 4 fragment having at least 10 contiguous amino acids in length corresponding to the polynucleotide region representing the junction resulting from the splicing of exon 6 to exon 9 of the BACE2 gene.

In some embodiments where, for example, BACE2sv1 polypeptides are used to develop antibodies that bind specifically to BACE2sv1 and not to other isoforms of BACE2, the BACE2sv1 polypeptides comprise at least 10 amino acids at the amino terminus of the BACE2sv1 polypeptide sequence having at least 10 contiguous amino acids in length corresponding to amino acids, including and downstream of, the amino terminal methionine of BACE2sv1. For example, the amino acid sequence: amino terminus-MQMCGAGLPV-carboxy terminus [SEQ ID NO 6], represents one embodiment of such an inventive BACE2sv1 polypeptide wherein a first 10 amino acid region is encoded by a nucleotide sequence starting with the “ATG” codon 70 nucleotides downstream of the 5′ end of exon 4 of the BACE2 gene.

In other embodiments where, for example, BACE2sv2 polypeptides are used to develop antibodies that bind specifically to BACE2sv2 and not to other isoforms of BACE2, the BACE2sv2 polypeptides comprise at least 10 amino acids of the BACE2sv2 polypeptide sequence corresponding to a junction polynucleotide region created by the alternative splicing of exon 6 to exon 9 of the primary transcript the BACE2 gene (see FIG. 1). For example, the amino acid sequence: amino terminus-ARASLKLQVL-carboxy terminus [SEQ ID NO 7] represents one embodiment of such an inventive BACE2sv2 polypeptide wherein a first 5 amino acid region is encoded by nucleotide sequence at the 3′ end of exon 6 of the BACE2 gene and a second 5 amino acid region is encoded by the nucleotide sequence directly after the novel splice junction. Preferably, at least 10 amino acids of the BACE2sv2 polypeptide comprises a first continuous region of 2 to 8 amino acids that is encoded by nucleotides at the 3′ end of exon 6 and a second continuous region of 2 to 8 amino acids that is encoded by nucleotides at the 5′ end of exon 9.

In other embodiments, BACE2sv1-specific antibodies are made using an BACE2sv1 polypeptide that comprises at least 20, 30, 40, or 50 amino acids of the BACE2sv1 sequence that corresponds to a polynucleotide region encoding amino acids, including and downstream of, the methionine codon located 70 nucleotides downstream of the 5′ end of exon 4 of the primary transcript of the BACE2 gene.

In other embodiments, BACE2sv2-specific antibodies are made using a BACE2sv2 polypeptide that comprises at least 20, 30, 40 or 50 amino acids of the BACE2sv2 sequence that corresponds to a junction polynucleotide region created by the alternative splicing of exon 6 to exon 9 of the primary transcript of the BACE2 gene. In each case the BACE2sv2 polypeptides are selected to comprise a first continuous region of at least 5 to 15 amino acids that is encoded by nucleotides at the 3′ end of exon 6 and a second continuous region of 5 to 15 amino acids that is encoded by nucleotides directly after the novel splice junction.

Antibodies to BACE2sv1 or BACE2sv2 have different uses, such as to identify the presence of BACE2sv1 or BACE2sv2, respectively, and to isolate BACE2sv1 or BACE2sv2 polypeptides, respectively. Identifying the presence of BACE2sv1 can be used, for example, to identify cells producing BACE2sv1. Such identification provides an additional source of BACE2sv1 and can be used to distinguish cells known to produce BACE2sv1 from cells that do not produce BACE2sv1. For example, antibodies to BACE2sv1 can distinguish human cells expressing BACE2sv1 from human cells not expressing BACE2sv1 or non-human cells (including bacteria) that do not express BACE2sv1. Such BACE2sv1 antibodies can also be used to determine the effectiveness of BACE2sv1 ligands, using techniques well known in the art, to detect and quantify changes in the protein levels of BACE2sv1 in cellular extracts, and in situ immunostaining of cells and tissues. In addition, the same above-described utilities also exist for BACE2sv2 specific antibodies.

Techniques for producing and using antibodies are well known in the art. Examples of such techniques are described in Ausubel, Current Protocols in Molecular Biology, John Wiley, 1987-1998; Harlow, et al., Antibodies, A Laboratory Manual, Cold Spring Harbor Laboratory, 1988; and Kohler, et al., 1975 Nature 256:495-7.

### BACE2sv1 and BACE2sv2 Binding Assays

A number of compounds have been synthesized to inhibit the aspartyl protease activity of BACE (Sinha, et.al., 1999, Nature 402, 537-540; Ghosh, et. al., 2000, J. Am. Chem. Soc. 122, 3522-3523). These compounds have been shown to also be effective inhibitors of BACE2 (Grüninger-Leitch, et. al., 2002, J. Biol. Chem. 277, 4687-4693). Methods for screening these compounds for their effects on the aspartyl protease activity of BACE and BACE2 have also been disclosed (see for example, Grüninger-Leitch, et. al., 2002, J. Biol. Chem. 277, 4687-4693). A person skilled in the art should be able to use these methods to screen BACE2sv1 or BACE2sv2 polypeptides for compounds that bind to, and in some cases functionally alter, the BACE2 isoform protein.

BACE2sv1, BACE2sv2, or fragments thereof, can be used in binding studies to identify compounds binding to or interacting with BACE2sv1, BACE2sv2, or fragments thereof, respectively. In one embodiment, the BACE2sv1, or a fragment thereof, can be used in binding studies with a BACE2 isoform protein, or a fragment thereof, to identify compounds that: bind to or interact with BACE2sv1 and other BACE2 isoforms; or bind to or interact with one or more other BACE2 isoforms and not with BACE2sv1. A similar series of compound screens can, of course, also be performed using BACE2sv2 rather than, or in addition to, BACE2sv1. Such binding studies can be performed using different formats including competitive and non-competitive formats. Further competition studies can be carried out using additional compounds determined to bind to BACE2sv1, BACE2sv2, or other BACE2 isoforms.

The particular BACE2sv1 or BACE2sv2 sequence involved in ligand binding can be identified using labeled compounds that bind to the protein and different protein fragments. Different strategies can be employed to select fragments to be tested to narrow down the binding region. Examples of such strategies include testing consecutive fragments about 15 amino acids in length starting at the N-terminus, and testing longer length fragments. If longer length fragments are tested, a fragment binding to a compound can be subdivided to further locate the binding region. Fragments used for binding studies can be generated using recombinant nucleic acid techniques.

In some embodiments, binding studies are performed using BACE2sv1 expressed from a recombinant nucleic acid. Alternatively, recombinantly expressed BACE2sv1 consists of the SEQ ID NO 2 amino acid sequence. In addition, binding studies are performed using BACE2sv2 expressed from a recombinant nucleic acid. Alternatively, recombinantly expressed BACE2sv2 consists of the SEQ ID NO 4 amino acid sequence.

Binding assays can be performed using individual compounds or preparations containing different numbers of compounds. A preparation containing different numbers of compounds having the ability to bind to BACE2sv1 or BACE2sv2 can be divided into smaller groups of compounds that can be tested to identify the compound(s) binding to BACE2sv1 or BACE2sv2, respectively.

Binding assays can be performed using recombinantly produced BACE2sv1 or BACE2sv2 present in different environments. Such environments include, for example, cell extracts and purified cell extracts containing a BACE2sv1 or BACE2sv2 recombinant nucleic acid; and also include, for example, the use of a purified BACE2sv1 or BACE2sv2 polypeptide produced by recombinant means which is introduced into different environments.

In one embodiment of the invention, a binding method is provided for screening for a compound able to bind selectively to BACE2sv1. The method comprises the steps: providing a BACE2sv1 polypeptide comprising SEQ ID NO 2; providing a BACE2 isoform polypeptide that is not BACE2sv1; contacting the BACE2sv1 polypeptide and the BACE2 isoform polypeptide that is not BACE2sv1 with a test preparation comprising one or more test compounds; and then determining the binding of the test preparation to the BACE2sv1 polypeptide and to the BACE2 isoform polypeptide that is not BACE2sv1, wherein a test preparation that binds to the BACE2sv1 polypeptide, but does not bind to BACE2 isoform polypeptide that is not BACE2sv1, contains one or more compounds that selectively binds to BACE2sv1.

In one embodiment of the invention, a binding method is provided for screening for a compound able to bind selectively to BACE2sv2. The method comprises the steps: providing a BACE2sv2 polypeptide comprising SEQ ID NO 4; providing a BACE2 isoform polypeptide that is not BACE2sv2; contacting the BACE2sv2 polypeptide and the BACE2 isoform polypeptide that is not BACE2sv2 with a test preparation comprising one or more test compounds; and then determining the binding of the test preparation to the BACE2sv2 polypeptide and to the BACE2 isoform polypeptide that is not BACE2sv2, wherein a test preparation that binds to the BACE2sv2 polypeptide, but does not bind to BACE2 isoform polypeptide that is not BACE2sv2, contains one or more compounds that selectively binds to BACE2sv2.

In another embodiment of the invention, a binding method is provided for screening for a compound able to bind selectively to a BACE2 isoform polypeptide that is not BACE2sv1. The method comprises the steps: providing a BACE2sv1 polypeptide comprising SEQ ID NO 2; providing a BACE2 isoform polypeptide that is not BACE2sv1; contacting the BACE2sv1 polypeptide and the BACE2 isoform polypeptide that is not BACE2sv1 with a test preparation comprising one or more test compounds; and then determining the binding of the test preparation to the BACE2sv1 polypeptide and the BACE2 isoform polypeptide that is not BACE2sv1, wherein a test preparation that binds the BACE2 isoform polypeptide that is not BACE2sv1, but does not bind the BACE2sv1, contains a compound that selectively binds the BACE2 isoform polypeptide that is not BACE2sv1. Alternatively, the above method can be used to identify compounds that bind selectively to a BACE2 isoform polypeptide that is not BACE2sv2 by performing the method with BACE2sv2 protein comprising SEQ ID NO 4.

The above-described selective binding assays can also be performed with a polypeptide fragment of BACE2sv2, wherein the polypeptide fragment comprises at least 10 consecutive amino acids that are coded by a nucleotide sequence that bridges the junction created by the splicing of the 3′ end of exon 6 to the 5′ end of exon 9. Similarly, the selective binding assays may also be performed using a polypeptide fragment of an BACE2 isoform polypeptide that is not BACE2sv2, wherein the polypeptide fragment comprises at least 10 consecutive amino acids that are coded by: a) a nucleotide sequence that is contained within exon 7 or 8 of the BACE2 gene; or b) a nucleotide sequence that bridges the junction created by the splicing of the 3′ end of exon 6 to the 5′ end of exon 7, the splicing of the 3′ end of exon 7 to the 5′ end of exon 8, or the splicing of the 3′ end of exon 8 to the 5′ end of exon 9, of the BACE2 gene.

### Aspartyl Protease Functional Assays

The identification of BACE2sv1 and BACE2sv2 as splice variants of BACE2 provides a means for screening for compounds that bind to BACE2sv1 and/or BACE2sv2 protein thereby altering the ability of the BACE2sv1 and/or BACE2sv2 polypeptide to bind to OM99-2 or any other inhibitor compound, or to perform enzymatic assay for aspartyl protease activity, including any BACE2 sub-reactions as described, for example by Grüninger-Leitch, et. al., (2002, J. Biol. Chem. 277, 4687-4693). Assays involving a functional BACE2sv1 or BACE2sv2 polypeptide can be employed for different purposes, such as selecting for compounds active at BACE2sv1 or BACE2sv2; evaluating the ability of a compound to effect aspartyl protease activity of each respective splice variant polypeptide; and mapping the activity of different BACE2sv1 and BACE2sv2 regions. BACE2sv1 and BACE2sv2 activity can be measured using different techniques such as: detecting a change in the intracellular conformation of BACE2sv1 or BACE2sv2; detecting a change in the intracellular location of BACE2sv1 or BACE2sv2; or measuring the level of aspartyl protease activity of BACE2sv1 or BACE2sv2.

Recombinantly expressed BACE2sv1 and BACE2sv2 can be used to facilitate determining whether a compound is active at BACE2sv1 and BACE2sv2. For example, BACE2sv1 and BACE2sv2 can be expressed by an expression vector in a cell line and used in a co-culture growth assay, such as described in WO 99/59037, to identify compounds that bind to BACE2sv1 and BACE2sv2. For example, BACE2sv1 can be expressed by an expression vector in a human kidney cell line 293 and used in a co-culture growth assay, such as described in U.S. patent application Ser. No. 20020061860, to identify compounds that bind to BACE2sv1. A similar strategy can be used for BACE2sv2.

Techniques for measuring aspartyl protease activity and substrate specificity are well known in the art (See Barrett, et. al., Proteolytic Enzymes: Aspartic and Metallo Peptidases, Methods in Enzymology, Vol. 248). In particular, Grüninger-Leitch, et. al., (2002, J. Biol. Chem. 277, 4687-4693) describe use of a Fluorescence Resonance Energy Transfer (FRET) assay to measure the affect of various inhibitors on the protease activity of BACE2. Yan. et. al., (2001, J. Biol. Chem. 276, 34019-34027) describe digestion of various peptide substrates with recombinant human BACE2 and quantifying the resultant products by reverse phase HPLC to determine substrate specificity. Other assays can also be used, such as ELISA (Vassar, et. al., 1999, Science 286, 735-741) and immunopreciptitaion (Lin, et. al., 2000, Proc. Natl Acad Scien 97, 1456-1460) to determine the effect of various BACE2 isoforms on the cleavage of APP.

BACE2sv1 or BACE2sv2 functional assays can be performed using cells expressing BACE2sv1 or BACE2sv2 at a high level. These proteins will be contacted with individual compounds or preparations containing different compounds. A preparation containing different compounds where one or more compounds affect BACE2sv1 or BACE2sv2 in cells over-producing BACE2sv1 or BACE2sv2 as compared to control cells containing expression vector lacking BACE2sv1 or BACE2sv2 coding sequences, can be divided into smaller groups of compounds to identify the compound(s) affecting BACE2sv1 or BACE2sv2 activity, respectively.

BACE2sv1 or BACE2sv2 functional assays can be performed using recombinantly produced BACE2sv1 or BACE2sv2 present in different environments. Such environments include, for example, cell extracts and purified cell extracts containing the BACE2sv1 or BACE2sv2 expressed from recombinant nucleic acid; and the use of a purified BACE2sv1 or BACE2sv2 produced by recombinant means that is introduced into a different environment suitable for measuring aspartyl protease activity.

### Modulating BACE2sv1 and BACE2sv2 Expression

BACE2sv1 or BACE2sv2 expression can be modulated as a means for increasing or decreasing BACE2sv1 or BACE2sv2 activity, respectively. Such modulation includes inhibiting the activity of nucleic acids encoding the BACE2 isoform target to reduce BACE2 isoform protein or polypeptide expressions, or supplying BACE2 nucleic acids to increase the level of expression of the BACE2 target polypeptide thereby increasing BACE2 activity.

### Inhibition of BACE2sv1 and BACE2sv2 Activity

BACE2sv1 or BACE2sv2 nucleic acid activity can be inhibited using nucleic acids recognizing BACE2sv1 or BACE2sv2 nucleic acid and affecting the ability of such nucleic acid to be transcribed or translated. Inhibition of BACE2sv1 or BACE2sv2 nucleic acid activity can be used, for example, in target validation studies.

A preferred target for inhibiting BACE2sv1 or BACE2sv2 is mRNA stability and translation. The ability of BACE2sv1 or BACE2sv2 mRNA to be translated into a protein can be effected by compounds such as anti-sense nucleic acid, RNA interference (RNAi) and enzymatic nucleic acid.

Anti-sense nucleic acid can hybridize to a region of a target mRNA. Depending on the structure of the anti-sense nucleic acid, anti-sense activity can be brought about by different mechanisms such as blocking the initiation of translation, preventing processing of mRNA, hybrid arrest, and degradation of mRNA by RNAse H activity.

RNAi also can be used to prevent protein expression of a target transcript. This method is based on the interfering properties of double-stranded RNA derived from the coding regions of gene that disrupts the synthesis of protein from transcribed RNA.

Enzymatic nucleic acids can recognize and cleave other nucleic acid molecules. Preferred enzymatic nucleic acids are ribozymes.

General structures for anti-sense nucleic acids, RNAi and ribozymes, and methods of delivering such molecules, are well known in the art. Modified and unmodified nucleic acids can be used as anti-sense molecules, RNAi and ribozymes. Different types of modifications can effect certain anti-sense activities such as the ability to be cleaved by RNAse H, and can effect nucleic acid stability. Examples of references describing different anti-sense molecules, and ribozymes, and the use of such molecules, are provided in U.S. Pat. Nos. 5,849,902; 5,859,221; 5,852,188; and 5,616,459. Examples of organisms in which RNAi has been used to inhibit expression of a target gene include: C. elegans (Tabara, et al., 1999, Cell 99, 123-32; Fire, et al., 1998, Nature 391, 806-11), plants (Hamilton and Baulcombe, 1999, Science 286, 950-52), Drosophila (Hammond, et al., 2001, Science 293, 1146-50; Misquitta and Patterson, 1999, Proc. Nat. Acad. Sci. 96, 1451-56; Kennerdell and Carthew, 1998, Cell 95, 1017-26), and mammalian cells (Bernstein, et al., 2001, Nature 409, 363-6; Elbashir, et al., 2001, Nature 411, 494-8).

### Increasing BACE2sv1 and BACE2sv2 Expression

Nucleic acids encoding for BACE2sv1 or BACE2sv2 can be used, for example, to cause an increase in BACE2 activity or to create a test system (e.g., a transgenic animal) for screening for compounds affecting BACE2sv1 or BACE2sv2 expression, respectively. Nucleic acids can be introduced and expressed in cells present in different environments.

Guidelines for pharmaceutical administration in general are provided in, for example, Remington's Pharmaceutical Sciences, 18^{th} Edition, supra, and Modern Pharmaceutics, 2^{nd} Edition, supra Nucleic acid can be introduced into cells present in different environments using in vitro, in vivo, or ex vivo techniques. Examples of techniques useful in gene therapy are illustrated in Gene Therapy & Molecular Biology. From Basic Mechanisms to Clinical Applications, Ed. Boulikas, Gene Therapy Press, 1998.

### EXAMPLES

Examples are provided below to further illustrate different features and advantages of the present invention. The examples also illustrate useful methodology for practicing the invention. These examples do not limit the claimed invention.

### Example 1

### Identification of BACE2sv1 and BACE2sv2 Using Microarrays

To identify variants of the “normal” splicing of the exon regions encoding BACE2, an exon junction microarray, comprising probes complementary to each splice junction resulting from splicing of the 9 exon coding sequences in BACE2 heteronuclear RNA (hnRNA), was hybridized to a mixture of labeled nucleic acid samples prepared from 44 different human tissue and cell line samples. Exon junction microarrays are described in PCT patent applications WO 02/18646 and WO 02/16650. Materials and methods for preparing hybridization samples from purified RNA, hybridizing a microarray, detecting hybridization signals, and data analysis are described in van't Veer, et al., (2002 Nature 415:530-536) and Hughes, et al., (2001 Nature Biotechnol. 19:342-7). Inspection of the exon junction microarray hybridization data (not shown) suggested that the structure of at least two of the exon junctions of BACE2 mRNA were altered in some of the tissues examined, suggesting the presence of at least two BACE2 splice variant mRNA populations within the “normal” BACE2 mRNA population. Reverse transcription and polymerase chain reactions (RT-PCR) were then performed using oligonucleotide primer sets complementary to exon 1 and exon 3, exon 2 and exon 4, exon 3 and exon 5, exon 4 and exon 6, and exon 6 and exon 9 of the “reference” BACE2 MRNA (NM_{—}012105) to confirm the exon junction array results and to allow the sequence structure of the splice variants to be determined.

### Example 2

### Confirmation of BACE2sv1 and BACE2sv2 Using RT-PCR

The structure of BACE2 mRNA in the regions spanning exons 1 to 9 was determined for a panel of human tissue and cell line samples using an RT-PCR based assay (data not shown). PolyA purified mRNA isolated from 44 different human tissue and cell line samples was obtained from BD Biosciences Clontech (Palo Alto, Calif.), Biochain Institute, Inc. (Hayward, Calif.), and Ambion Inc. (Austin, Tex.). RT-PCR primers were selected that were complementary to sequences in exons 1, 2, 3, 4, 5, 6, and 9 of the reference exon coding sequence in BACE2 mRNA (NM_{—}012105). In some of the samples, no amplification product (amplicon) was observed with the use of primer sets spanning exons 1 and 3, and exons 2 and 4. However, amplicons were observed in these samples with the use of primer sets spanning exons 3 and 5 and exons 4 and 6. These results suggested a truncated protein, with the coding sequence of exons 1 and 2 of the reference BACE2 mRNA (NM_{—}012105) missing.

The EST geneseqn:AAD09473 human aspartyl protease Asp 1 (patent application WO 01/46398 A2) contains sequence complimentary to nucleotide sequence spanning the intron 2/exon 3 boundary of the BACE2 gene. A forward primer was designed that was complementary to BACE2 intron 2 sequence, and the reverse primer was designed to be complementary to exon 4 of the BACE2 mRNA sequence (NM_{—}012105). The BACE2 intron 2 forward primer has the sequence: 5′ GTCTACCCTGGTACCGCCTTTCTTT 3′ [SEQ ID NO 8]; and the BACE2 exon 4 reverse primer has the sequence: 5′ CTCCACACATCTGCAT GGAGAAAAC 3′ [SEQ ID NO 9]. Given that the “reference” BACE2 mRNA does not contain any intron sequence, amplification with the intron 2 and exon 4 primer set (hereinafter BACE2₂₄ primer set) is expected to yield no RT-PCR product representing the “reference” BACE2 mRNA.

The BACE2 exon 6 forward primer has the sequence: 5′ TATAACGCAGACA AGGCCATCGTGGA 3′ [SEQ ID NO 10]; and the exon 9 reverse primer has the sequence: 5′ GCAGCAGGACGATTAAGACAAGGAGGAT 3′ [SEQ ID NO 11]. Based upon the nucleotide sequence of BACE2 mRNA, the BACE2 exon 6 and exon 9 primer set (hereinafter BACE2₆₋₉ primer set) was expected to amplify a 589 base pair amplicon representing the region corresponding to exons 6 to 9 of the “reference” BACE2 mRNA.

Twenty-five ng of polyA mRNA from each tissue was subjected to a one-step reverse transcription-PCR amplification protocol using the Qiagen, Inc. (Valencia, Calif.), One-Step RT-PCR kit, using the following conditions:

Cycling conditions were as follows:

50° C. for 30 minutes;

95° C. for 15 minutes;

35 cycles of:

94° C. for 30 seconds;

63.5° C. for 40 seconds;

72° C. for 50 seconds; then

72° C. for 10 minutes.

RT-PCR amplification products (amplicons) were size fractionated on a 2% agarose gel. Selected amplicon fragments were manually extracted from the gel and purified with a Qiagen Gel Extraction Kit. Purified amplicon fragments were sequenced from each end (using the same primers used for RT-PCR) by Qiagen Genomics, Inc. (Bothell, Wash.).

An RT-PCR amplicon of about 408 basepairs was obtained from a number of human mRNA samples using the BACE2₂₋₄ primer set. At least two different RT-PCR amplicons were obtained from human mRNA samples using the BACE2₆₋₉ primer set. Every human tissue and cell line assayed exhibited the expected amplicon size of 589 base pairs for normally spliced BACE2 mRNA. However, in addition to the expected BACE2 amplicon of 589 base pairs, some tissues exhibited an amplicon of about 270 base pairs, and others also very faintly showed an additional amplicon of about 270 base pairs.

Sequence analysis of the about 408 base pair amplicon, herein referred to as “BACE2sv1,” revealed that this amplicon results from the deletion of exons 1 and 2 of the BACE2 hnRNA and the retention of intron 2 sequence upstream and adjacent to exon 3, forming a novel 5′ exon. Sequence analysis of the about 270 base pair amplicon, herein referred to as “BACE2sv2,” revealed that this amplicon results from the splicing of exon 6 of the BACE2 hnRNA to exon 9; that is, the exon 7 and 8 coding sequence is completely absent. Thus, the RT-PCR results confirmed the junction probe microarray data reported in Example 1, which suggested that BACE2 mRNA is composed of a mixed population of molecules wherein in at least two of the BACE2 mRNA splice junctions is altered. The results are summarized in Table 1 below.

TABLE 1

| Sample | BACE2sv1 | BACE2sv2 |
|---|---|---|
| Heart | X | |
| Kidney | | |
| Liver | | |
| Brain | | |
| Placenta | | |
| Lung | | |
| Fetal Brian | | |
| Leukemia Promyelocytic (HL-60) | | |
| Adrenal Gland | | |
| Fetal Liver | | |
| Salivary Gland | | |
| Pancreas | | |
| Skeletal Muscle | | |
| Brain Cerebellum | | |
| Stomach | | X |
| Trachea | | |
| Thyroid | | |
| Bone Marrow | | |
| Brain Amygdala | | |
| Brain Caudate Nucleus | | |
| Brain Corpus Callosum | | |
| Ileocecum | | |
| Lymphoma Burkitt's (Raji) | | |
| Spinal Cord | | |
| Lymph Node | | |
| Fetal Kidney | | X |
| Uterus | | |
| Spleen | | X |
| Brain Thalamus | | |
| Fetal Lung | | X |
| Testis | | X |
| Melanoma (G361) | | X |
| Lung Carcinoma (A549) | | |
| Adrenal Medula, normal | | |
| Brain, Cerebral Cortex, normal; | | |
| Descending Colon, normal | | X |
| Prostate | | |
| Duodenum, normal | | X |
| Epididymus, normal | | |
| Brain, Hippocamus, normal | | |
| Ileum, normal | | |
| Interventricular Septum, normal | X | |
| Jejunum, normal | | |
| Rectum, normal | | |

### Example 3

### Cloning of BACE2sv1 and BACE2sv2

Microarray and RT-PCR data indicate that in addition to the normal BACE2 reference mRNA sequence, (NM_{—}012105), encoding BACE2 protein, (NP_{—}036237), two novel splice variant forms of BACE2 mRNA also exist in a number of tissues.

A full length BACE2 clone having nucleotide sequence comprising the splice variants identified in Example 2 (hereafter referred to as BACE2sv1 and BACE2sv2) are isolated using a 5′ “forward” BACE2 primer and a 3′ “reverse” BACE2 primer, to amplify and clone the entire BACE2sv1 or BACE2sv2 mRNA coding sequences, respectively. The 5′ “forward” BACE2sv1 primer designed for isolation of full length clones corresponding to the BACE2sv1 splice variant has the nucleotide sequence of 5′ ATGCAGATGTGTGGAGCCGGCTTGCCC 3′ [SEQ ID NO 12]. The 3′ “reverse” BACE2sv1 primer is designed to have the nucleotide sequence of 5′ TCATTTCCAGCGATGTCTGACCAGAGA 3′ [SEQ ID NO 13]. The 5′ “forward” BACE2sv2 primer designed for isolation of full length clones corresponding to the BACE2sv2 splice variant has the nucleotide sequence of 5′ ATGGGCGCACTGGCCCGGGC GCTGCTG 3′ [SEQ ID NO 14]. The 3′ “reverse” BACE2sv2 primer is designed to have the nucleotide sequence of 5′ CTACATCCTCTGTTGAGAAAGGCCCGG 3′ [SEQ ID NO 15].

RT-PCR

The BACE2sv1 and BACE2sv2 cDNA sequences are cloned using a combination of reverse transcription (RT) and polymerase chain reaction (PCR). More specifically, about 25 ng of human spleen, in the case of BACE2sv1, and human stomach, in the case of BACE2sv2, polyA mRNA (Palo Alto, Calif.) is reverse transcribed using Superscript II (Gibco/Invitrogen, Carlsbad, Calif.) and oligo d(T) primer (RESGEN/Invitrogen, Huntsville, Ala.) according to the Superscript II manufacturer's instructions. For PCR, 1 μl of the completed RT reaction is added to 40 μl of water, 5 μl of 10×buffer, 1 μl of dNTPs and 1 μl of enzyme from the Clontech (Palo Alto, Calif.) Advantage 2 PCR kit. PCR is done in a Gene Amp PCR System 9700 (Applied Biosystems, Foster City, Calif.) using the BACE2 “forward” and “reverse” primers. After and initial 94° C. denaturation of 1 minute, 35 cycles of amplification are performed using a 30 second denaturation at 94° C. followed by a 40 second annealing at 63.5° C. and a 50 second synthesis at 72° C. The 35 cycles of PCR are followed by a 10 minute extension at 72° C. The 50 μl reaction is then chilled to 4° C. 10 μl of the resulting reaction product is run on a 1% agarose (Invitrogen, Ultra pure) gel stained with 0.3 μg/ml ethidium bromide (Fisher Biotech, Fair Lawn, N.J.). Nucleic acid bands in the gel are visualized and photographed on a UV light box to determined if the PCR has yielded products of the expected size, in the case of the predicted BACE2sv1 and BACE2sv2 mRNAs, products of about 870 and 1041 bases, respectively. The remainder of the 50 μl PCR reactions from human spleen and stomach is purified using the QIAquik Gel extraction Kit (Qiagen, Valencia, Calif.) following the QIAquik PCR Purification Protocol provided with the kit. About 50 μl of product obtained from the purification protocol is concentrated to about 6 μl by drying in a Speed Vac Plus (SC 110 A, from Savant, Holbrook, N.Y.) attached to a Universal Vacuum Sytem 400 (also from Savant) for about 30 minutes on medium heat.

### Cloning of RT-PCR Products

About 4 T1 of the 6 T1 of purified BACE2sv1 and BACE2sv2 RT-PCR products from human spleen and stomach, respectively, are used in a cloning reaction using the reagents and instructions provided with the TOPO TA cloning kit (Invitrogen, Carlsbad, Calif.). About 2 T1 of the cloning reaction is used following the manufacturer's instructions to transform TOP 10 chemically competent E. coli provided with the cloning kit. After the 1 hour recovery of the cells in SOC medium (provided with the TOPO TA cloning kit), 200 Ti of the mixture is plated on LB medium plates (Sambrook, et al., in Molecular Cloning, A Laboratory Manual, 2^{nd} Edition, Cold Spring Harbor Laboratory Press, 1989) containing 100 Tg/ml Ampicillin (Sigma, St. Louis, Mo.) and 80 Tg/ml X-GAL (5-Bromo-4-chloro-3-indoyl B-D-galactoside, Sigma, St. Louis, Mo.). Plates are incubated overnight at 37° C. White colonies are picked from the plates into 2 ml of 2×LB medium. These liquid cultures are incubated overnight on a roller at 37° C. Plasmid DNA is extracted from these cultures using the Qiagen (Valencia, Calif.) Qiaquik Spin Miniprep kit. Twelve putative BACE2sv1 and BACE2sv2 clones, respectively, are identified and prepared for a PCR reaction to confirm the presence of the expected BACE2sv1 exon 4 to exon 9 and BACE2sv2 exon 6 to exon 9 splice variant structures. A 25 T1 PCR reaction is performed as described above (RT-PCR section) to detect the presence of BACE2sv1, except that the reaction includes miniprep DNA from the TOPO TA/ BACE2sv1 ligation as a template. An additional 25 T1 PCR reaction is performed as described above (RT-PCR section) to detect the presence of BACE2sv2, except that the reaction includes miniprep DNA from the TOPO TA/ BACE2sv2 ligation as a template. About 10 T1 of each 25 T1 PCR reaction is run on a 1% Agarose gel and the DNA bands generated by the PCR reaction are visualized and photographed on a UV light box to determine which minipreps samples have PCR product of the size predicted for the corresponding BACE2sv1 and BACE2sv2 splice variant mRNAs. Clones having the BACE2sv1 structure are identified based upon amplification of an amplicon band of 870 basepairs. Clones having the BACE2sv2 structure are identified based upon amplification of an amplicon band of 1041 basepairs, whereas a normal reference BACE2 clone would give rise to an amplicon band of 1557 basepairs. DNA sequence analysis of the BACE2sv1 or BACE2sv2 cloned DNAs confirm a polynucleotide sequence representing the absence of exons 1-3 plus 69 nucleotides of exon 4 in the case of BACE2sv1, and the deletion of exons 7 and 8 in the case of BACE2sv2.

The polynucleotide sequence of BACE2sv1 mRNA (SEQ ID NO 1) encodes a BACE2sv1 protein (SEQ ID NO 2), similar to the reference BACE2 protein (NP_{—}036237), but lacking the first 229 amino acids of the reference BACE2 protein (NP_{—}036237) due to utilization of a translation initiation AUG codon downstream from the AUG initiation codon utilized by the reference BACE2 protein (NP_{—}036237).

The polynucleotide sequence of BACE2sv2 mRNA (SEQ ID NO 3) contains an open reading frame that encodes a BACE2sv2 protein (SEQ ID NO 4) similar to the reference BACE2 protein (NP_{—}036237), but lacking amino acids encoded by exons 7 and 8 of the full length coding sequence of reference BACE2 mRNA (NM_{—}012105). The alternative splicing of exon 6 to exon 9 not only deletes a 319 base pair region corresponding to exons 7 and 8, but also results in a protein reading frame shift at the novel exon 6/exon 9 splice junction, creating a protein translation reading frame that is out of alignment in comparison to the reference BACE2 protein reading frame. This shift in reading frame creates a premature termination codon, resulting in the production of an altered and shorter BACE2sv2 protein as compared to the reference BACE2 protein (NP_{—}036237).

All patents, patent publications, and other published references mentioned herein are hereby incorporated by reference in their entireties as if each had been individually and specifically incorporated by reference herein. While preferred illustrative embodiments of the present invention are shown and described, one skilled in the art will appreciate that the present invention can be practiced by other than the described embodiments, which are presented for purposes of illustration only and not by way of limitation. Various modifications may be made to the embodiments described herein without departing from the spirit and scope of the present invention. The present invention is limited only by the claims that follow.

What is claimed:

1. A purified human nucleic acid comprising SEQ ID NO 3, or the complement thereof.

2. The purified nucleic acid of claim 1, wherein said nucleic acid comprises a region encoding SEQ ID NO 4.

3. The purified nucleic acid of claim 1, wherein said nucleotide sequence encodes a polypeptide consisting of SEQ ID NO 4.

4. A purified polypeptide comprising SEQ ID NO 4.

5. The polypeptide of claim 4, wherein said polypeptide consists of SEQ ID NO 4.

6. An expression vector comprising a nucleotide sequence encoding SEQ ID NO 4, wherein said nucleotide sequence is transcriptionally coupled to an exogenous promoter.

7. The expression vector of claim 6, wherein said nucleotide sequence encodes a polypeptide consisting of SEQ ID NO 4.

8. The expression vector of claim 6, wherein said nucleotide sequence comprises SEQ ID NO 3.

9. The expression vector of claim 6, wherein said nucleotide sequence consists of SEQ ID NO 3.

10. A method for screening for a compound able to bind to BACE2sv2 comprising the steps of:
(a) expressing a polypeptide comprising SEQ ID NO 4 from recombinant nucleic acid;
(b) providing to said polypeptide a test preparation comprising one or more test compounds;
(c) and measuring the ability of said test preparation to bind to said polypeptide.

11. The method of claim 10, wherein said steps (b) and (c) are performed in vitro.

12. The method of claim 10, wherein said steps (a), (b), and (c) are performed using a whole cell.

13. The method of claim 10, wherein said polypeptide is expressed from an expression vector.

14. The method of claim 10, wherein said polypeptide consists of SEQ ID NO 4.

15. A method of screening for compounds able to bind selectively to BACE2sv2 comprising the steps of:
(a) providing a BACE2sv2 polypeptide comprising SEQ ID NO 4;
(b) providing one or more BACE2 isoform polypeptides that are not BACE2sv2;
(c) contacting said BACE2sv2 polypeptide and said BACE2 isoform polypeptide that is not BACE2sv2 with a test preparation comprising one or more compounds; and
(d) determining the binding of said test preparation to said BACE2sv2 polypeptide and to said BACE2 isoform polypeptide that is not BACE2sv2, wherein a test preparation which binds to said BACE2sv2 polypeptide, but does not bind to said BACE2 polypeptide that is not BACE2sv2, contains a compound that selectively binds said BACE2sv2 polypeptide.

16. The method of claim 15, wherein said BACE2sv2 polypeptide is obtained by expression of said polypeptide from an expression vector comprising a polynucleotide encoding SEQ ID NO 4.

17. The method of claim 16, wherein said polypeptide consists of SEQ ID NO 4.

18. A method for screening for a compound able to bind to or interact with a BACE2sv2 protein or a fragment thereof comprising the steps of:
(a) expressing a BACE2sv2 polypeptide comprising SEQ ID NO 4 or fragment thereof from a recombinant nucleic acid;
(b) providing to said polypeptide a labeled BACE2 ligand that binds to said polypeptide and a test preparation comprising one or more compounds; and
(c) measuring the effect of said test preparation on binding of said labeled BACE2 ligand to said polypeptide, wherein a test preparation that alters the binding of said labeled BACE2 ligand to said polypeptide contains a compound that binds to or interacts with said polypeptide.

19. The method of claim 18, wherein said steps (b) and (c) are performed in vitro.

20. The method of claim 18, wherein said steps (a), (b) and (c) are performed using a whole cell

21. The method of claim 18, wherein said polypeptide is expressed from an expression vector

22. The method of claim 18, wherein said BACE2sv2 ligand is an aspartyl protease inhibitor.

23. The method of claim 21, wherein said expression vector comprises SEQ ID NO 3 or a fragment of SEQ ID NO 3.

24. The method of claim 21, wherein said polypeptide comprises SEQ ID NO 4 or a fragment of SEQ ID NO 4.
